(19) United States
(12) Patent Application Publication
(10) Pub. No.: US 2004/0005261 A1
(43) Pub. Date: Jan. 8, 2004

(54) Plasma sterilization apparatus

(76) Inventor: Jung-Suek Ko, Seoul (KR)

Correspondence Address: MANELLI DENISON & SELTER PLLC, 7th Floor, 2000 M Street, N.W., Washington, DC, 20036-3307, US

(21) Appl. No.: 10/419,926

(22) Filed: Apr. 22, 2003

(30) Foreign Application Priority Data
Apr. 23, 2002 (KR) 10-2002-0022320
Mar. 17, 2003 (KR) 10-2003-0016588

(51) Int. Cl. A61L 2/00; B01J 19/08

(52) U.S. Cl. 422/305; 422/186; 422/22

(57) ABSTRACT

A plasma sterilization apparatus to prevent color change or hardening of materials, e.g., polymers, by generating plasma in a separate plasma generation chamber and supplying it into a sterilization chamber containing an article to be sterilized. A plasma generation chamber includes electrodes installed in close proximity to each other, preferably separated by 0.5-40 cm, to generate plasma with high density even with a small capacity high frequency power source. The electrodes are connected to a high frequency power source to generate the plasma. An injection heater evaporates an aqueous hydrogen peroxide solution used as a microcidal agent, and injects the resulting vapor along with air into the plasma generation chamber. A sterilization chamber communicates with the plasma generation chamber at a remote position through a control valve. A vacuum pump is connected to the sterilization chamber, to form a vacuum state in the sterilization chamber.

### BACKGROUND OF THE INVENTION

1. Field of the Invention

The present invention relates to a plasma sterilization apparatus for killing any microorganisms present on the surfaces of articles to be sterilized, e.g., medical instruments, using plasma in a gaseous state. More particularly, the present invention relates to a plasma sterilization apparatus for sterilizing an article to be sterilized by supplying plasma previously generated in a separate plasma generation chamber into a remote sterilization chamber.

2. Background of Related Art

Various methods have been developed and utilized to sterilize various types of disposable or recycled medical instruments. Sterilization techniques using high temperature and high pressure were widely used in the past. Also, ethylene oxide (EtO) gas has been employed in this regard. Recently, plasma sterilization systems using hydrogen peroxide have received much attention.

However, there are disadvantages with the conventional sterilization methods. The methods using high temperature and high pressure are difficult to utilize in connection with articles easily affected by heat or steam. The use of ethylene oxide (EtO) gas can result in severe environmental damage due to the use of CFC gas, a major source of environmental pollution, as a carrier gas. Moreover, the use of ethylene oxide gas requires a long aeration time to remove the gas remaining in articles after sterilization.

A plasma sterilization system using hydrogen peroxide, in which sterilization is achieved using (1) a sterilization apparatus comprised of a vacuum pump for injecting an aqueous hydrogen peroxide solution that serves as a vaporized microbiocidal agent into a sterilization chamber to generate plasma, and (2) a radio frequency generator for the generation of a radio frequency electrode and an essential radio frequency signal, is disclosed in Korean Pat. No. 132233.

However, such a plasma sterilization method employing hydrogen peroxide is problematic in the following respects. Since plasma is generated at the electrodes (between the cathode and the anode) of the sterilization chamber containing the article to be sterilized, the article comes into direct contact with the plasma, causing physical and chemical changes to polymer-based medical devices, e.g., color change or hardening.

In addition, when over 70% of the sterilization chamber contains an article to be sterilized, the article is not completely sterilized because contact between the article and the plasma is not completely achieved. Moreover, the sterilization chamber is limited in that sterilization is not always uniform depending on the size of the articles to be sterilized.

In particular, when an electrode field is created at the electrodes (between the cathode and the anode) of the sterilization chamber by supplying power through a radio frequency generator, the article placed near the cathode of the sterilization chamber is often incompletely sterilized due to the concentration of electrons at the cathode.

In addition, all of the U.S. patents owned by the Abtox company, USA (U.S. Pat. Nos. 5,084,239, 5,244,629, 5,413,758, 5,645,796, 6,261,518, etc.) disclose plasma-generating systems, each of which is comprised of a sterilization chamber connected directly to a plasma generation chamber. Also, each of these systems employ a plasma generator using a microwave source to generate plasma electrodelessly.

In the Abtox patents, since the plasma generator generates plasma electrodelessly, the plasma generation chamber and the sterilization chamber should be installed separately. In addition, since the microwave plasma is generated at the plasma generator using a high frequency of 2.45 GHz, there is a high potential for ultraviolet (UV) radiation being emitted from the plasma, thereby requiring an additional device or object to shield the UV radiation, and high costs with respect to industrial application.

### SUMMARY OF THE INVENTION

To solve the problems encountered in the prior art, the present invention aims to provide a plasma sterilization apparatus for sterilizing an article to be sterilized; which is capable of preventing color changes or hardening to the material of the article, e.g., polymers; which is free from the influence of the self-bias voltage phenomenon; and which achieves sterilization by generating plasma in a separate plasma generation chamber and supplying the plasma into a sterilization chamber containing the article.

It is another object of the present invention to provide a plasma sterilization apparatus comprised of a plasma generation chamber capable of reducing the interval between electrodes regardless of the article to be sterilized, thus allowing the miniaturization of the invention and facilitation of plasma generation, even when using a high frequency power source of small capacity. The present invention also avoids limitations in the size of the sterilization chamber because the plasma is generated at a high density at the narrow interval between the electrodes of the plasma generation chamber, thus allowing the maximum volume of the article to be sterilized to be increased.

To achieve the above objects, the present invention provides a plasma sterilization apparatus comprised of a sterilization chamber for receiving therein an article to be sterilized; a plasma generation chamber located at a remote position that communicates with the sterilization chamber and has two electrodes therein; a vacuum pump connected to the sterilization chamber, which is capable of extracting air and creating a vacuum state in both the sterilization chamber and the plasma generation chamber; a high frequency power source connected to one (cathode) of the two electrodes of the plasma generation chamber through both an impedance matching controller and an impedance matching circuit; and an injection heater that communicates with the plasma generation chamber and is useful in the evaporation of microbiocidal agents for the generation of plasma and the injection of the evaporated product into the plasma generation chamber.

In the plasma generation chamber, the two electrodes are installed in close proximity to each other at an interval of 0.5-40 cm to generate plasma at a high density even with a high frequency power source of small capacity.

To control the internal pressure of both the sterilization chamber and the plasma generation chamber, an automatic pressure control valve is installed between the sterilization chamber and the plasma generation chamber, and another is installed between the sterilization chamber and the vacuum pump.

### BRIEF DESCRIPTION OF THE DRAWINGS

The above objects, features and other advantages of the present invention will be more clearly understood from the following detailed description taken in conjunction with the accompanying drawing.

FIG. 1 is a schematic view of a plasma sterilization apparatus according to an embodiment of the present invention.

### DETAILED DESCRIPTION OF ILLUSTRATIVE EMBODIMENTS

The plasma sterilization apparatus according to the present invention will now be described in detail in conjunction with the accompanying drawing.

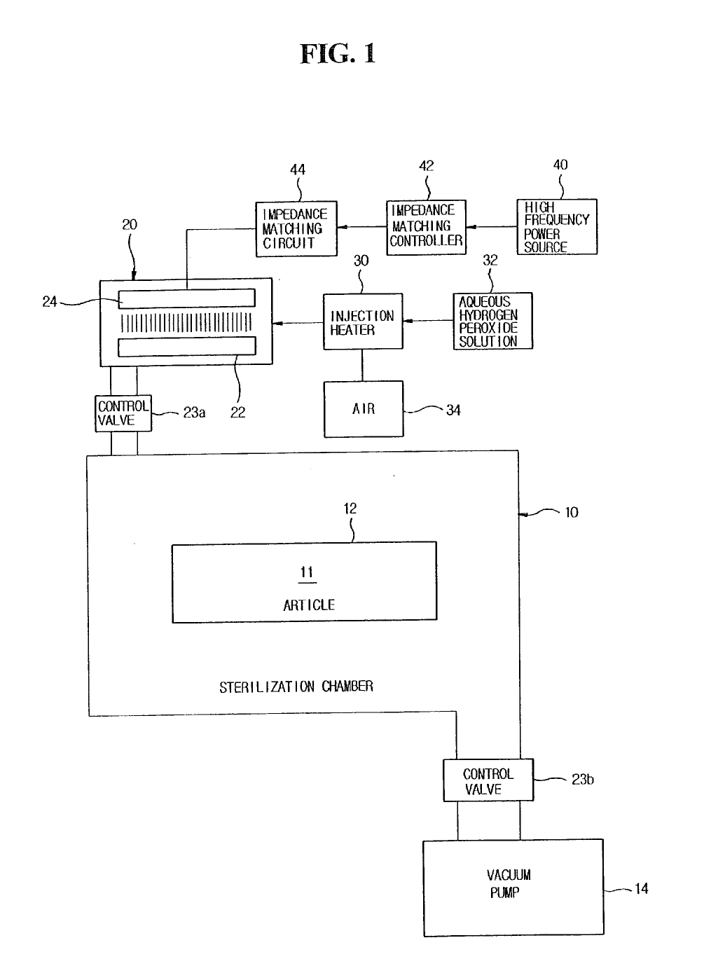

FIG. 1 contains the schematics of a plasma sterilization apparatus according to the present invention, where the microorganisms present on the surface of an article 11 to be sterilized, e.g., a medical instrument, are killed using air 34 and an aqueous hydrogen peroxide solution 32 as agents for the generation of plasma, where the aqueous hydrogen peroxide solution is useful as a precursor of the active species and is mixed with air to generate plasma. In the present invention, a sterilization process employing oxidizing power is adopted. For this reason, air 34 is used to generate the mixed gas to be used as a microbiocidal agent, in which the mixed gas is generated by evaporating the aqueous hydrogen peroxide solution 32 and mixing the resulting vapor with air 34 by an injection heater 30.

In other words, since hydrogen peroxide has only 85% of the oxidizing power of ozone, a much higher oxidizing power is obtained by combining the oxidizing power of hydrogen peroxide and the oxidizing power of air.

Fluorine, the hydroxyl group and ozone all have higher oxidizing powers than hydrogen peroxide. However, fluorine has a very high level of corrosiveness and toxicity, and the hydroxyl group and ozone do not exist in a stable state in nature, thus making their use impossible.

The sterilization chamber 10 is a chamber capable of containing an article 11, e.g., a medical instrument or a surgical operation tool, which is wrapped in a wrapper 12. The chamber is connected to a vacuum pump 14 to create a vacuum state in the sterilization chamber 10 using air extracted from the inside of the sterilization chamber 10.

Plasma from the plasma generation chamber 20 is communicated to the sterilization chamber 10 through a control valve 23a, to supply the reactive species of the plasma into the sterilization chamber 10. The plasma generation chamber 20 includes two electrodes, i.e., an anode 22 and a cathode 24. The plasma generation chamber 20 is remotely separated from the sterilization chamber 10 via a pathway including the control valve 23a, with plasma being sucked into the sterilization chamber 10 due to a vacuum created by the vacuum pump 14, also remote from the sterilization chamber 10 via another control valve 23b.

Since the plasma generation chamber 20 does not directly come into contact with the article to be sterilized, it is possible for the cathode 24 to be located near the anode 22 at an interval of 0.5-40 cm, thereby allowing for the generation of plasma with a high density using high frequency power source 40 having a small capacity.

When the interval between the cathode 24 and the anode 22 is zero, an electrical short circuit occurs, resulting in no generation of plasma. When the interval between the cathode 24 and the anode 22 is below 0.5 cm, plasma is easily generated, but the resulting high temperature of the electrodes causes damage to the electrodes and the low temperature of the plasma is raised. When the interval between the cathode 24 and the anode 22 is over 40 cm, excessive power is required to generate plasma and the required equipment must be larger, thereby resulting in higher production costs.

In addition, the plasma generation chamber 20 is connected to the injection heater 30 for the purposes of evaporating the aqueous hydrogen peroxide solution 32, which serves as a microbiocidal agent, to generate plasma with uniform density, and heat-injecting the resulting vapor along with air 34 thereinto.

The high frequency power source 40 having a frequency capable of generating plasma in an optimal state is connected to the cathode 24 of the plasma generation chamber through both the impedance matching controller 42 and the impedance matching circuit 44. The frequency of the high frequency power source 40 may include a variety of frequency bands. The higher the frequency, the higher the density of the plasma. However, high frequency requires more expensive equipment and additional equipment capable of shielding electromagnetic radiation. Therefore, it is preferable to select a frequency band suitable to equipment currently in use.

The automatic pressure control valves 23a and 23b are located between the plasma generation chamber 20 and the sterilization chamber 10, and between the sterilization chamber 10 and the vacuum pump 14, respectively.

An article to be sterilized may be sterilized by the plasma sterilization apparatus according to the present invention, as follows. An article 11 to be sterilized, e.g., a medical instrument or a surgical tool, is wrapped in the wrapper 12, and placed into the sterilization chamber 10, and the door of the sterilization chamber 10 is closed. At this point, after opening the automatic pressure control valves 23a and 23b, the vacuum pump 14 connected to the sterilization chamber 10 is operated and extracts air from the chamber, forming a vacuum in the sterilization chamber 10 and the plasma generation chamber 20 at the desired pressure.

After the vacuum chamber 14 creates a vacuum inside the sterilization chamber 10 and the plasma generation chamber 20 at the desired vacuum pressure level, the injection heater 30 forms a mixed gas containing the microbiocidal agents, i.e., the aqueous hydrogen peroxide solution 32 and the air 34. This mixed gas is injected into the plasma generation chamber 20. Herein, the pressure of the mixed gas is controlled by the automatic pressure control valves 23a and 23b, located between the plasma generation chamber 20 and the sterilization chamber 10 and between the sterilization chamber 10 and the vacuum pump 14, respectively.

After the plasma generation chamber 20 reaches the desired reaction pressure level through the injection of the mixed gas of the aqueous hydrogen peroxide solution 32 and air 34, i.e., the microbiocidal agent, high frequency power from the power source 40 is applied to the cathode 24 of the sterilization chamber 10 through the impedance matching circuit 44 and the impedance controller 42. Because of the high frequency power applied to the cathode 24 in the sterilization chamber 10, plasma having a high density is generated between the cathode 24 and the anode 22 in the sterilization chamber 10.

The high frequency power source 40 uses a frequency of 13.56 MHz. As UV radiation is not emitted from a plasma generated by such a low frequency, there is no requirement for an additional device or object to shield the UV radiation.

Herein, the high frequency power source 40 generates plasma having a low temperature below 100° C. using a pulsed high frequency power, where the power is applied intermittently to the power source 40. The pulsed application of high frequency power prevents the overheating of the gas within the sterilization chamber 10 and the overheating of the article 11 to be sterilized.

The reactive species in the high density plasma generated as described above uniformly diffuse from the plasma generation chamber 20 to the sterilization chamber 10, maintaining a desired plasma atmosphere. The reactive species diffused to the sterilization chamber 10 interact with the article 11 to sterilize the article.

Herein, the plasma within the sterilization chamber 10 is transferred thereinto after being generated by the plasma, generation chamber 20. Therefore, the temperature of the inside of the sterilization chamber 10 is lower than that of the plasma generation chamber 20.

The atmosphere of the inside of the sterilization chamber 10 depends on the supplied electric power of the high frequency power source 40 applied to the cathode 24 of the plasma generation chamber 20 and the concentration of the mixed gas, which is generated by the evaporation of the aqueous hydrogen peroxide solution 32 and air 34. Sterilization is finished within the short time period of approximately 5 min from the start of the plasma generation. As the sterilization process finishes within such a short time, it is preferable to continuously maintain the desired plasma atmosphere for a predetermined time to achieve sufficient sterilization of the article.

The sterilization efficiency in the sterilization chamber 10 depends on the concentration of the mixed gas produced by the evaporation of the aqueous hydrogen peroxide solution 32 and air 34, i.e., the microbiocidal agents. However, since the sterilization efficiency also depends on the supplied electric power, electric power is applied to achieve optimal sterilization efficiency.

The wrapper 12 is used to wrap the article 11 to be sterilized before entry into the sterilization chamber 10. Therefore, the wrapper is selected from materials that are not reactive to the plasma atmosphere and that possesses a fiber-like structure capable of allowing ventilation of plasma therethrough.

As described above, the article 11 is completely sterilized by continuously maintaining the plasma atmosphere for a predetermined length of time.

After sterilization is complete, the high frequency power source 40 is cut off and the automatic pressure control valves 23a and 23b are opened. At this time, the vacuum pump 14 is operated to sufficiently exhaust the mixed gas (comprised of the hydrogen peroxide vapor and air) in the sterilization chamber 10. Then, the sterilization chamber 10 is returned to normal atmospheric pressure and the sterilized wrapped article 11 is removed from the sterilization chamber 10.

When applied to sterilize articles such as medical instruments or surgical tools, unlike the conventional sterilization system using ethylene oxide gas, the sterilization apparatus of the present invention does not produce toxic by-products during plasma generation from the hydrogen peroxide and air used as microbiocidal agents. Rather the by-products degrade into non-toxic substances, avoiding the need for an additional device to remove residual hydrogen peroxide on the article 11 to be sterilized or on the wrapper 12.

### EXPERIMENTAL EXAMPLE 1

In this test, the sterilization efficiency of the plasma sterilization apparatus of the present invention was evaluated by comparing it with a single-body type plasma sterilization apparatus in which a plasma generation chamber and a sterilization chamber are integrated into a single chamber and plasma generation and sterilization occur in the single chamber.

Bacillus stearothermophilus (spore No. 2.04×10⁶) was used as a test microorganism, which is a biological indicator (BI) commercially available under the name “Cyclesure” produced by a certain company in the U.S. and used as a sterilization indicator for clinical instruments in many hospitals.

The BI was introduced into the plasma sterilization apparatus of the present invention, where plasma generation and sterilization are performed separately in different chambers, and the single-body type plasma sterilization apparatus, where plasma generation and sterilization occur in the single chamber. Sterilization was carried out at the optimal conditions for each sterilization apparatus. Thereafter, the collected BI samples were incubated at 55° C. for up to 72 hrs in an incubator and the color of the BI samples was analyzed. The results are shown in Table 1 below, in which 50 BI samples per sterilization apparatus were analyzed.

TABLE 1

| | Plasma | No. of Samples | No. of Samples |
|---|---|---|---|
| Microbiocidal | Sterilization | Successfully | Not Successfully |
| Agent | Apparatus | Sterilized* | Sterilized** |
| H₂O₂ | Single-body type | 50 | 0 |
| | apparatus | | |
| H₂O₂/air | Apparatus of the | 50 | 0 |
| | present invention | | |

*Bl samples display negative response (no color change)
**Bl samples display positive response (color changed)

As shown in Table 1, the plasma sterilization apparatus of the present invention, where plasma generation and sterilization are performed separately in different chambers, was found to have the same sterilization efficiency as in the conventional plasma sterilization apparatus, where plasma generation and sterilization occur in a single chamber. In addition, sterilization was successful in both of the plasma sterilization apparatus of the present invention, which employs a mixed gas of hydrogen peroxide and air, and the prior art, which utilizes only hydrogen peroxide. The sterilization apparatus of the present invention used a mixed gas composed of hydrogen peroxide and air as the microbiocidal agents, where sterilization was achieved by oxidation, and the microbiocidal agents may be typically selected according to their oxidizing power.

### EXPERIMENTAL EXAMPLE 2

After sterilization occurred using the plasma sterilization apparatus of the present invention, the polymer-like materials of the articles were investigated for color change and hardening.

Sterilization was performed according to the same method as in Experimental Example 1, except that polyethylene (PE) was used as a specimen to identify whether the plasma sterilization apparatus of the present invention induces color change and hardening. The specimen PE was about 0.5 mm thick and about 40 mm in both width and length. After sterilization, the appearance of the collected specimens was evaluated using LUCI 100 (spectral two-beam calorimeter), a type of color measurement equipment in which color is measured in three kinds of specimens: specimen 1-PE not exposed to a sterilization procedure; specimen 2-PE exposed to a sterilization procedure by the single-body type plasma sterilization apparatus; and specimen 3-PE exposed to a sterilization procedure by the plasma sterilization apparatus of the present invention.

TABLE 2

| Specimen | Reactive | Plasma Sterilization | Color Difference |
|---|---|---|---|
| No. | Gas | Apparatus | (5 tests) |
| 1 | — | — | Control |
| 2 | H₂O₂ | Single-body type | Wavelength deviation |
| | | apparatus | (Δλ): about 160 nm |
| 3 | H₂O₂/air | Apparatus of the present | Wavelength deviation |
| | | invention | (Δλ): about 60 nm |

As shown in Table 2 above, when specimens 2 and 3, which had undergone sterilization by the single-body type plasma sterilization apparatus using only hydrogen peroxide and the plasma sterilization apparatus of the present invention using a mixed gas of hydrogen peroxide vapor and air, respectively, were compared to the control specimen 1, they were found to have wavelength deviations of about 160 nm and about 60 nm, respectively.

These results demonstrate that specimen 2, which was exposed to the sterilization procedure of the single-body type plasma sterilization apparatus, underwent a greater color change than specimen 3, which was exposed to the sterilization procedure of the plasma sterilization apparatus of the present invention. Therefore, it is believed that because the plasma generation chamber is installed separately from the sterilization chamber in the plasma sterilization apparatus of the present invention, an article to be sterilized is not directly influenced by the plasma generated at the electrodes of the plasma generation chamber.

### EXPERIMENTAL EXAMPLE 3

The plasma sterilization apparatus of the present invention was evaluated for volumetric sterilization capacity, and compared to the single-body type plasma sterilization apparatus.

To investigate the sterilization capacity of the sterilization chamber of the plasma sterilization apparatus of the present invention and the single-body type plasma sterilization apparatus, 70 Suction Catheters, 70 Nelaton Catheters and 70 Balloon Catheters were wrapped in Sterilization Pouches along with the same BI sample as in Experimental Example 1 and used as specimens. When the volume of a sterilization apparatus is 100% full, the single-body type plasma sterilization apparatus typically has a maximum sterilization rate of 70%. However, for experimental purposes, about 80% of the single-body type plasma sterilization apparatus was filled with the specimens (168 specimens were placed on the upper and lower trays for sterilization). On the other hand, 95% of the sterilization chamber of the plasma sterilization apparatus of the present invention was filled with the specimens (200 specimens were placed on the upper and lower trays for sterilization). Then, sterilization was performed under the optimal conditions for both apparatus and the collected BI samples were incubated at 55° C. for up to 72 hrs in an incubator. The results of the test, which was repeated ten times, are provided in Table 3 below.

TABLE 3

| | | | No. of | No. of |
|---|---|---|---|---|
| | | Plasma | Samples | Samples Not |
| Microbiocidal | Filling | Sterilization | Successfully | Successfully |
| Agent | Volume (%) | Apparatus | Sterilized* | Sterilized** |
| H₂O₂ | 80 | Single- | 0 | 10 |
| | | body type | | |
| | | apparatus | | |
| H₂O₂/air | 95 | Apparatus | 10 | 0 |
| | | of the | | |
| | | present | | |
| | | invention | | |

As shown in Table 3, upon comparing the results of the test using the single-body type plasma sterilization apparatus employing only hydrogen peroxide with the results of the test using the plasma sterilization apparatus employing a mixed gas of hydrogen peroxide and air, it was found that the plasma sterilization apparatus of the present invention, unlike the single-body type apparatus, may be filled to sterilize more articles at a time.

This advantage seems to result from the fact that in the plasma sterilization apparatus of the present invention, the plasma generation chamber having the two electrodes is separated from the sterilization chamber through a control valve.

As already described herein, the plasma sterilization apparatus of the present invention first generates plasma in a separate plasma generation chamber and then supplies the plasma into a sterilization chamber containing an article to be sterilized, thereby exposing the article to the plasma, making it possible to prevent color change or hardening of materials such as polymers, and being immune to the self-bias voltage phenomenon.

In addition, the plasma sterilization apparatus is comprised of a plasma generation chamber capable of reducing the interval between its electrodes, thus allowing for its miniaturization irregardless of the article to be sterilized and facilitating the generation of plasma even using a small capacity high frequency power source. Further, the plasma sterilization apparatus is not limited in terms of the size of its sterilization chamber because the plasma is generated at high density by the narrow interval between the electrodes of the plasma generation chamber, thus allowing the maximum volume of the article to be sterilized to be increased.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1. A plasma sterilization apparatus, comprising:
a sterilization chamber for receiving therein an article to be sterilized;
a plasma generation chamber located at a separate position from the sterilization chamber, said plasma generation chamber being in communication with the sterilization chamber, and said plasma generation chamber including two electrodes therein;
a vacuum pump connected to the sterilization chamber, the vacuum pump being capable of extracting air from the sterilization chamber to form a vacuum state in both the sterilization chamber and the plasma generation chamber;
a high frequency power source connected to one (cathode) of the two electrodes of the plasma generation chamber through both an impedance matching controller and an impedance matching circuit; and
an injection heater that communicates with the plasma generation chamber to evaporate microbiocidal agents for the generation of plasma and the injection of the evaporated product into the plasma generation chamber.

2. The plasma sterilization apparatus according to claim 1, wherein:
the two electrodes of the plasma generation chamber are installed in close proximity to each other at an interval of between 0.5 cm and 40 cm to generate plasma having a high density even with a high frequency power source of small capacity.

3. The plasma sterilization apparatus according to claim 1, wherein:
to control internal pressure of both the sterilization chamber and the plasma generation chamber, an automatic pressure control valve is installed between the plasma generation chamber and the sterilization chamber, and another control valve is installed between the sterilization chamber and the vacuum pump.

4. A plasma sterilization apparatus, comprising:
a plasma generation chamber including two electrodes with a close interval of between about 0.5 cm and 40 cm;
a sterilization chamber separated from receiving plasma from the plasma generation chamber via a plasma source control valve, the sterilization chamber being adapted to accommodate an article to be sterilized; and
a vacuum pump connected to the sterilization chamber, the vacuum pump being capable of extracting air from the sterilization chamber to form a vacuum state in the sterilization chamber.

5. The plasma sterilization apparatus according to claim 4, further comprising:
a high frequency power source feeding to one of the two electrodes of the plasma generation chamber; and
an injection heater in communication with the plasma generation chamber, the injection heater functioning to evaporate microbiocidal agents, and injecting the evaporated product into the plasma generation chamber.

6. The plasma sterilization apparatus according to claim 5, further comprising:
an impedance matching controller and an impedance matching circuit between the high frequency power source and the plasma generation chamber.

7. The plasma sterilization apparatus according to claim 4, further comprising:
a vacuum pump control valve installed between the sterilization chamber and the vacuum pump to allow control of internal pressure of the sterilization chamber, via the vacuum pump control valve, and internal pressure of the plasma generation chamber, via the plasma source control valve.

8. The plasma sterilization apparatus according to claim 5, wherein:
the high frequency power source uses a frequency of about 13.56 MHz.
